(19) United States
(12) Patent Application Publication
(10) Pub. No.: US 2007/0207517 A1
(43) Pub. Date: Sep. 6, 2007

(54) Breast milk ethanol screening system and method

(76) Inventors: Julie K. Jumonville, Austin, TX (US); Elizabeth Smith, Austin, TX (US)

Correspondence Address: H. DALE LANGLEY, JR.;THE LAW FIRM OF H. DALE LANGLEY, JR. PC, 610 WEST LYNN, AUSTIN, TX, 78703, US

(21) Appl. No.: 11/361,590

(22) Filed: Feb. 24, 2006

(51) Int. Cl. C12Q 1/26

(52) U.S. Cl. 435/25

(57) ABSTRACT

A test kit detects presence of ethanol alcohol in human breast milk. The test kit includes an alcohol oxidase reactive in the presence of ethanol alcohol in breast milk and oxygen, an indicator having a visible color when not subjected to oxidizing agent, in sufficient amount for visible observance, and a peroxidase reactive in the presence of acetaldehyde and peroxide, to yield oxidizing agent, in sufficient amount to induce change to the visible color of the hydrogen donor indicator. The test kit is a sealed pack containing a test strip. The test strip is for one-time use, and disposable after testing. The test strip is handled by a tester, typically a breast-feeding mother. The mother contacts her breast milk lactate with testing portion of the test strip. The testing portion selectively reacts to alcohol, and not other components of breast milk. A reactive agent of the testing portion of the test strip changes color in the presence of alcohol. The mother observes the result to determine if there is presence of alcohol in the breast milk.

### BACKGROUND OF THE INVENTION

The present invention generally relates to ethanol screen testing and, more particularly, relates to screening for ethanol in breast milk of lactating females prior to breastfeeding.

Various dangers of excessive ethanol alcohol consumption by humans, typically in the form of alcoholic beverages, are known and publicized. Research and reporting continues. The predominant focus of research and reporting has typically been the effects of such consumption on the particular consumer. Of course, health concerns, legal requirements and restraints, and other similar issues have often been raised, particularly in heavy alcohol consumers.

Research of alcohol consumption by mothers during pregnancy, and impact to the in utero infant, has received much attention. Less attention has tended to be directed, however, to post-partum consumption of alcohol by breast-feeding mothers. In recent years, studies of breast milk composition and nutritional values of breast-feeding, post-birth maternal alcohol use and effects on lactation and hormones involved in breast-feeding, and infant developmental and other effects of breast-feeding have been documented.

Presently, it is widely held that human milk is uniquely superior to alternatives for infant feeding, and breast-feeding generally appears quite beneficial for both the mother and the infant. Breastfeeding and the Use of Human Milk, American Academy of Pediatrics, Work Group on Breastfeeding (1997). Of course, breast-feeding presents concerns for the mother, in that dietary, intake, drug, and other effects can be passed through to the feeding infant. This can constrain breast-feeding mothers from normal social lifestyles and require various dietary and activity constraints or practices. It also can cause consternation and worry to the mother. One major concern of breast-feeding mothers is whether or not alcohol consumption by the mother can adversely or otherwise affect the infant.

Folklore and traditional wisdom (prior to at least as recent as about 1998, and even continuing to an extent thereafter until very recently) has been that some alcohol consumption, particularly of beer and wine, by breast-feeding mothers may be beneficial for increased lactate production, initiation of breastfeeding, infant nutrition, and other enhancement of breastfeeding success. Alcohol's Effect on Lactation, Alcohol Res. Health, 25(3):230-4 (Mennella, et al., 2001); Beer and Breastfeeding, Adv. Exp. Med. Biol., 478:23-8 (Koletko, et al., 2000); Breastfeeding & Drugs: Alcohol, http://www.breastfeedingbasics.org/cgi-bin/deliver.cgi/content/Drugs/alcohol.html (O'Connor, 1998)(moderate alcohol use on infant development is inconclusive; occasional alcoholic drink by lactating woman probably fine); Alcohol and breastfeeding, J. Hum Lact., 11(4); 321-323 (Anderson, 1995)(alcohol believed to stimulate breast milk production; may stimulate sucking initially); Infants' suckling responses to the flavor of alcohol in mothers' milk, Dev. Psychobiol., 26(8):459-66 (Mennella, 1993); Transfer of alcohol to human milk, New Eng. J. Med., 325(14):981-985 (Mennella, et al., 1991)(limited amount of alcohol believed transferred to mothers' milk); Beer, breastfeeding, and folklore, N. Engl. J. Med, 321(7):425-30 (Menella, et al., 1989).

Studies now show that alcohol presence in mothers' milk may, in fact, have significant effects on breast-feeding and breast-fed infants. For example, infants may nurse more frequently but consume significantly less breast milk if it contains alcohol [Beer, breastfeeding, and folklore, N. Engl. J. Med., 321(7):425-30 (Menella, et al., 1989)]; infant motor development may be adversely affected if exposed to breast milk alcohol [Alcohol, Breastfeeding, and Development at 18 Months, Pediatrics 2002; 209; 72 (Little, et al., 2002); Maternal alcohol use during breast-feeding and infant mental and motor development at one year, N. Engl. J. Med., 322(5):338-9 (Little, et al., 1990)]; lactate production by drinking mothers is not increased, and may decrease [Lactation and alcohol: clinical and nutritional effects, Arch. Latinoam Nutr., 54(1):25-35 (de Araujo Burgos, et al., 2004); Alcohol's Effect on Lactation, Alcohol Res. Health, 25(3):230-4 (Mennella, 2001)]; infant sleep patterns may change with intake of breast milk containing alcohol [Sleep disturbances after acute exposure to alcohol in mothers' milk, Alcohol, 25(3):153-8 (Mennella, et al., 2001); Effects of Exposure to Alcohol in Mother's Milk on Infant Sleep, Pediatrics, 101(5):e2 (Menella, et al., 1998)]; and other problems to the infant and/or the mother.

In light of these new indications (observed primarily only since after about of 1989 and more so in even more recent years in the 21^{st} century) of potential harms of alcohol in breast-feeding, it appears advantageous to avoid breast-feeding if alcohol is present in breast milk. Authorities indicate that time studies on time to zero level of alcohol in breast milk are dependent on a number of factors, particularly including the amount of alcohol consumed by and the body weight of the mother. Alcohol and Breast Feeding: Calculation of Time to Zero Level in Milk, Biol. Neonate, 80:219-22 (Ho, et al., 2001)(nomogram of weight and number of drinks to estimated time to zero from this source is provided in Appendix A). These same authorities recommend that the breast-feeding mother try to delay breast feeding to allow complete elimination of alcohol from her breast milk. Id. Notwithstanding the time to zero estimates presently available (as in the foregoing source), that source recognizes that the values may be faster or slower in some women, depend on many factors beyond weight and amount of alcohol consumed, and can only be estimated based on average results in the studies.

For at least 20 years or so (i.e., since as early as about 1985), various tests and test kits for individual use have been proposed for testing alcohol levels in saliva and urine. More accurate measures of alcohol concentrations in the body after consuming ethanol alcohol beverages, however, generally require clinical diagnostic blood tests. The tests and test kits for individual use have been limited to determination of bodily alcohol levels to avoid illegal driving and the like. There have not been any individual-use alcohol tests or test kits for purposes of gauging for breastfeeding. Of course, breast milk levels of alcohol may not be well reflected from saliva, urine, and/or blood tests, as breast milk is produced through bodily processes of the mother that can significantly differ from the bodily processes for saliva, urine, and blood.

It would therefore be advantageous to provide individual-use tests and test kits for directly detecting alcohol presence in breast milk. Moreover, levels of alcohol contained in breast milk may/may not be important to the ramifications of breast-feeding. Therefore, it would be advantageous to provide such tests and test kits that detect alcohol presence alone. Of course, compositional levels of alcohol in breast milk could also be considered important for the determinations, in certain instances. So, such tests and test kits could additionally/alternatively beneficially provide for alcohol level determinations in the milk. Objectives of individual-use tests and test kits in these regards could provide substantial health and other benefits.

It would, therefore, be a significant improvement in the art and technology to provide tests and test kits for detection of alcohol in breast milk of breast-feeding mothers. The present invention provides numerous advantages and improvements, including improvements and nuances in the foregoing respects.

### SUMMARY OF THE INVENTION

An embodiment of the invention is a test reagent for detecting alcohol. The test reagent includes alcohol oxidase reactive in the presence of alcohol in breast milk and oxygen, to yield acetaldehyde and peroxide, hydrogen donor indicator having a visible color when not subjected to oxidizing agent, in sufficient amount for visible observance, and peroxidase reactive in the presence of acetaldehyde and peroxide, to yield oxidizing agent, in sufficient amount to induce change to the visible color of the hydrogen donor indicator.

Another embodiment of the invention is a method of testing breast milk lactate for presence of alcohol. The method includes obtaining the lactate, contacting a reactive agent with the lactate, the reactive agent being selective to alcohol versus components for breast milk not including alcohol, and observing a result of the step of contacting.

Yet another embodiment of the invention is a method of testing of human breast milk for presence of ethanol alcohol. The method includes lactating to obtain a breast milk lactate sample, contacting a reactive agent with the sample, the reactive agent being selective to alcohol versus components for human breast milk not including alcohol, and observing a result of the step of contacting.

Another embodiment of the invention is a test kit for detecting presence of alcohol in human breast milk. The test kit includes a substrate test strip, an alcohol detector of the strip, and a visible agent for changing color in the presence of alcohol on the alcohol detector.

Yet another embodiment of the invention is a test kit for detecting presence of ethanol alcohol in human breast milk. The test kit includes alcohol oxidase reactive in the presence of ethanol alcohol in breast milk and oxygen, indicator having a visible color when not subjected to oxidizing agent, in sufficient amount for visible observance, and peroxidase reactive in the presence of acetaldehyde and peroxide, to yield oxidizing agent, in sufficient amount to induce change to the visible color of the hydrogen donor indicator.

Another embodiment of the invention is a method of testing human breast milk for presence of ethanol alcohol. The method includes providing a sealed pack containing a test strip, the test strip having a reactive component and a handling component, opening the sealed pack, handling the test strip via the handling component, lactating, contacting the breast milk with the reactive component of the test strip, and observing a result from the step of contacting.

### BRIEF DESCRIPTION OF THE DRAWINGS

The present invention is illustrated by way of example and not limitation in the accompanying figures, in which like references indicate similar elements, and in which:

FIG. 1 illustrates a perspective view of a test strip for detecting presence of alcohol in breast milk of a breast-feeding mother, according to certain embodiments of the invention;

FIG. 2 illustrates a side view of the test strip of FIG. 1, including a carrier strip and a reactive agent on the carrier strip, according to certain embodiments of the invention;

FIG. 3 illustrates a side view of an alternate test strip, similar to that of FIG. 1 but having a pool drop of reactive agent on an end of the carrier strip, according to certain embodiments of the invention;

FIG. 4 illustrates a side view of another test strip, including reactive agent affixed to an end of the carrier strip, according to certain embodiments of the invention;

FIG. 5 illustrates a side view of another alternate test strip, including a carrier strip that is impregnable or otherwise carries interstitially therewith a reactive agent, according to certain embodiments of the invention;

FIG. 6 illustrates a side of yet another test strip, the test strip including a carrier vehicle at an end of the strip, the carrier vehicle serving to absorb or hold a reactive agent, according to certain embodiments of the invention;

FIG. 7 illustrates a perspective view of a test kit, including the test strip of FIG. 1, the test kit having a sealed enclosure pack containing the test strip and a desiccant, according to certain embodiments of the invention;

FIG. 8 illustrates a front view of an alternate test kit, having a sealed enclosure containing a reactive agent of a carrier strip, with a handling portion of the carrier strip protruding from within the sealed enclosure, according to certain embodiments of the invention;

FIG. 9 illustrates a perspective view of a bottle test kit, including a vial and cap, the cap having a test piece extending therethrough with a reactive agent of the test piece contained within the vial and cap enclosure, according to certain embodiments of the invention;

FIG. 10 illustrates a perspective view of another bottle test kit, a vial and cap enclosure containing a reactive agent, a separate handling strip is provided and includes or is formed of a material to carry the reactive agent when dipped into the vial into the agent, according to certain embodiments of the invention;

FIG. 11 illustrates a side perspective view of another alternative test kit, including an enclosed packet of reactive agent, the enclosed packet being semi-permeable to permit soaking of the reactive agent contents with breast milk applied thereto, and providing for externally visible observance of test results with the reactive agent, according to certain embodiments of the invention; and

FIG. 12 illustrates a method for testing a breast milk lactate for presence of alcohol, using a test strip, according to certain embodiments of the invention.

### DETAILED DESCRIPTION

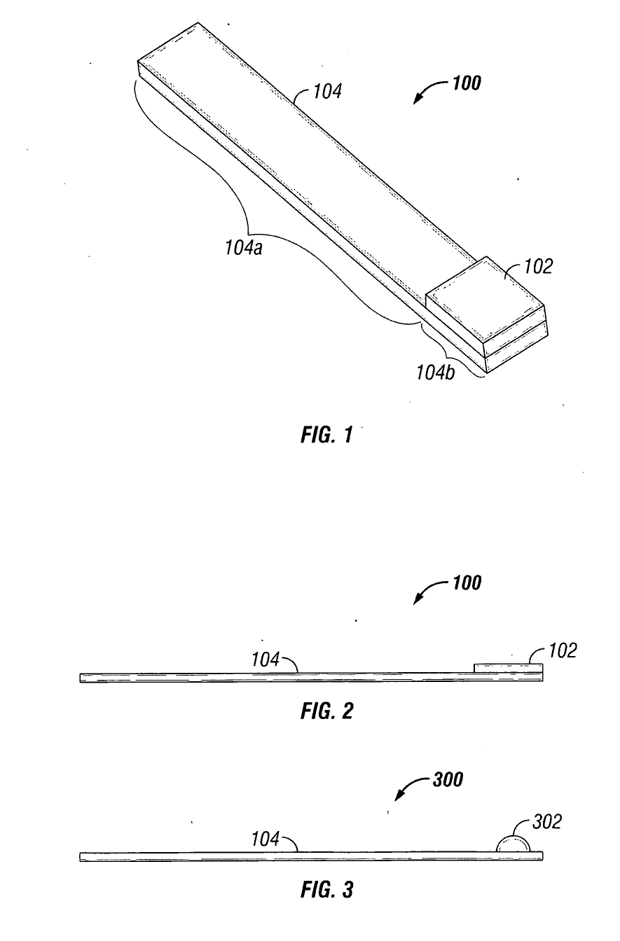

Referring to FIG. 1, a test strip 100 comprises a reactive agent 102 maintained on a carrier strip 104. The carrier strip 104 has a handling portion 104a and a sampling portion 104b. The handling portion 104a of the carrier strip 104 is for human handling of the test strip 100 via that portion. The indicator agent 102 is connected on, impregnated in, or otherwise maintained at the sampling portion 104b of the carrier strip 104.

The carrier strip 104 is a paper, plastic, wood, or similar material. The carrier strip 104 is suitable for maintaining the indicator agent 102 of the test strip 100 and also for non-interference and non-reaction in testing operations utilizing the test strip 100 as later described herein. The test strip 100 can be slim and linear, as in FIG. 1, or can have other shape that generally provides a portion for handling by a human user and another portion for location of an agent for testing. For example, a stick or round pole structure, a triangular or square slip, or other dimensioned piece can be suitable. In many (but not necessarily all) instances, the agent's properties for testing can be affected or otherwise disturbed if allowed to touch skin, become exposed to anything other than liquids in testing, such as, for example, exposure to air, gases, water, sweat, oils, emulsions, non-test liquids, and/or be subjected to certain temperature, pressure or other extremes; therefore, human handling is usually best limited to the handling portion 104a of the carrier strip 104.

The reactive agent 102 is an ethanol alcohol recognition composition or material. For example, the reactive agent 102 is reactive with ethanol alcohol if and when the reactive agent 102 is exposed to the alcohol in testing operations. Any of a variety of compounds or materials can be suitable as the reactive agent 102. The reactive agent 102 is itself adhereable, impregnable, wickable, stainable, or otherwise connectable in, with or to the carrier strip 104 at the sampling portion 104b. Alternately, a glue, attacher, or sticky matter is employed to retain the reactive agent 102 with the carrier strip 104 at the sampling portion 104b, provided, such matter must not affect or alter reactivity of or tests with the reactive agent 102 and must not be itself be reactive inconsistent with the reactive agent 102.

Referring to FIG. 2, the test strip 100 of FIG. 1 includes the carrier strip 104. The reactive agent 102 is maintained on a surface of the carrier strip 104. The reactive agent 102 is generally layered and attached to the carrier strip 104 at the sampling portion 104b.

Referring to FIG. 3, an alternate test strip 300, includes the carrier strip 104, or similar support, to that of the test strip 100 of FIG. 1. An alternate reactive agent 302 is a drop of matter reactive to ethanol alcohol. The reactive agent 302 is similarly disposed at the sampling portion 104b of the carrier strip 104. The reactive agent 302 can be hardened material, affixed in, with or to the carrier strip 104, or otherwise thereon disposed, including in liquid, gel or other physical form.

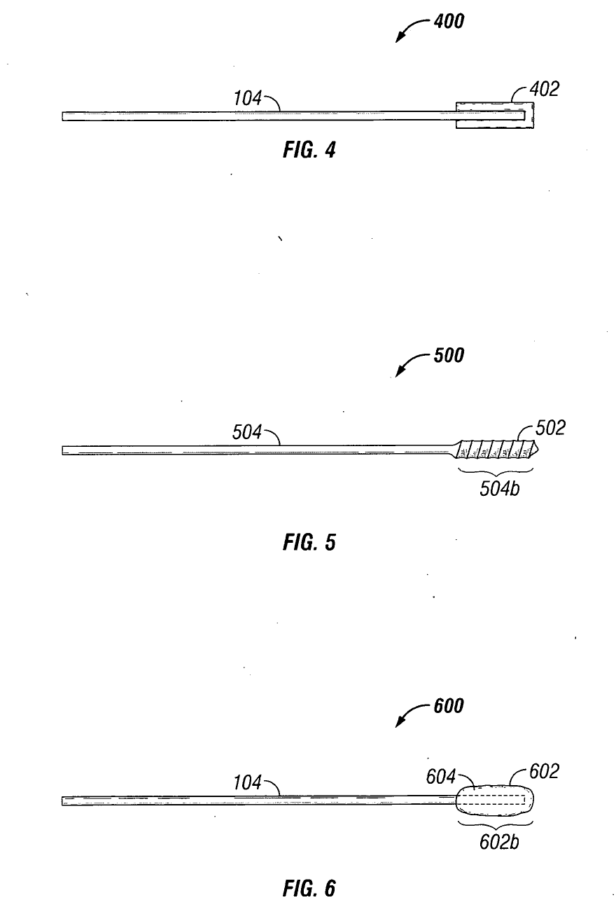

Referring to FIG. 4, another alternate test strip 400 also includes the carrier strip 104 or similar support. The carrier strip 104 has reactive agent 402 located on at least two sides of the carrier strip 104 at the sampling portion 104b. The sampling portion 104b is formed with the reactive agent 402, for example, by dipping the sampling portion 104b into the reactive agent 402, such as in manufacture where the reactive agent 402 is of a liquid form that hardens and attaches after dipping. Alternately, a cap-type form of reactive agent 402 can be attached to the sampling portion 104b at the end thereof. In FIG. 4, the reactive agent 402 is illustrated as formed on three sides at the sampling portion 104b (i.e., on a top side, a bottom side and an end); however, edges can also include the reactive agent 402, the reactive agent 402 can be disposed only on the top side and bottom side, or otherwise.

Referring to FIG. 5, another test strip 500 includes a carrier strip 504 that is a permeable, semi-permeable, wicking, blotting, or similar material. The reactive agent 502 is contained within interstices of the material of the carrier strip 504 at the sampling portion 504b. The reactive agent 502 is liquid, dried liquid, powder, or hardened matter that is soaked up at least in part by the material of the carrier strip 504, for example, during manufacture, just prior to test initiation, or otherwise as may be applicable for the particular agent employed for testing.

Referring to FIG. 6, another alternate test strip 600 includes the carrier strip 104, such as that of FIG. 1, and the carrier strip has an end carrier vehicle 602. The carrier vehicle 602 is a cotton, sponge, or similar material that can retain therein or thereon a reactive agent 604. The carrier vehicle 602 is formed, placed or joined to the carrier strip 104 at the sampling portion 602b. The reactive agent 604 is maintained by the carrier vehicle 602 with the test strip 600, for testing via the reactive agent 604 in the carrier vehicle 602.

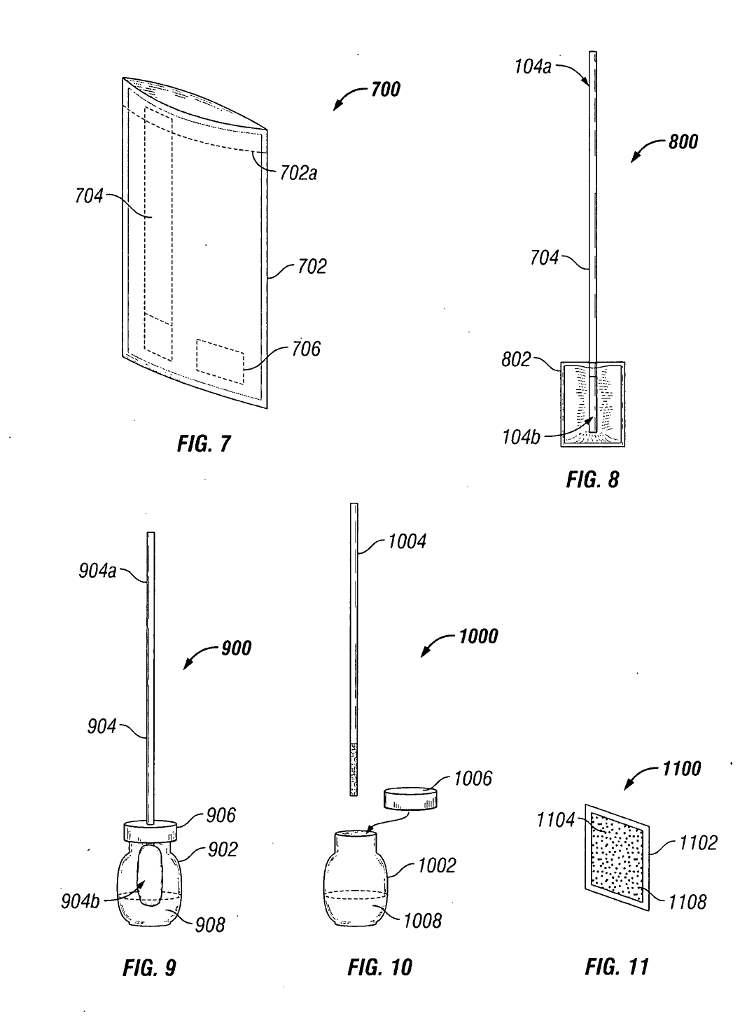

Referring to FIG. 7, a test kit 700 includes a package 702. The package 702 is a sealed, tearable enclosure, sufficient for containing a test kit strip 704 (substantially of the form of the test strips previously described, including a carrier strip and reactive agent). The package 702 is, for example, an environmentally sealed envelope, such as a metallic, plastic, paper or otherwise impermeable or semi-permeable material. The package 702 can be vacuum sealed or otherwise enclosed within a sterile (or, in certain instances, at least clean and generally clear lab or manufacturing facility) environment, wherein the test kit strip 704 is included prior to complete enclosure. The package 702 can, but need not necessarily, include tear dimples or other similar packaging aspects that enable ease of tearing or cutting to remove the test kit strip 704. In every event, the test kit strip 704 is protected by the package 702, once enclosed therein, from external matter that could contaminate, complicate, or otherwise affect testing via the test kit strip 704. The package 702 can further include other elements within the package 702, as desired, for example, desiccant 706, other matters to aid preservation or veracity, testing instructions, contents descriptions, regulatory or other warnings and notices, and other matters.

In use, the package 702 is opened, the test kit strip 704 is retrieved therefrom, and testing via the test kit strip 704 can be conducted.

Referring to FIG. 8, an alternate test kit 800 includes an alternate package 802 and the test kit strip 704. A sampling portion 104b (substantially as described with respect to the other Figures) of the test kit strip 704 is enclosed within the package 802, and the handling portion 104a thereof protrudes through the enclosure of the package 802. For example, the package 802 is shrink-wrapped or formed around the sampling portion 104b, in a manner permitting the handling portion 104a (or part thereof) to extend outside the package 802. In the package 802, the enclosure is at least fairly completely sealed against or formed to or with the handling portion 104a, to prevent contamination or other testing concerns to reagent for testing of the sampling portion 104b. The package 802 can be folded back to expose the sampling portion 104b when testing is desired, or, as applicable, may be removed from the test kit strip 704.

Referring to FIG. 9, another alternate test kit 900 includes a vial 902. The vial 902 is sealed by a cap 906, such as a screw-on/off top or other sealing cap. A vial test strip 904 of the kit 900 includes a handling portion 904a and a sampling portion 904b. The test strip 904 is formed to extend through the cap 906 with minimal clearance sufficient to prevent exposure of contents of the vial 902 when the cap 906 is in place to seal the enclosure. Alternately, the test strip 904 is integral to the cap 906, and is either formed therewith, as part thereof, or is secured therewith extending through the cap 906. The handling portion 904a extends from the cap 906 outside the enclosure of the vial 902 and cap 906. Internally in that enclosure, the sampling portion 904b is disposed, extending from the cap 906 into the vial 902 (and, if and to the extent applicable, some length of extension of the handling portion 904a as may be necessary to cause the sampling portion 904b to sit in desired location within the vial 902). The vial 902 and cap 906 are each any of a glass, plastic, metal or similar rigid material sufficient to protect, and not react with, reagent of the sampling portion 904b.

In certain embodiments, a test reagent 908 (shown in phantom) can fill the vial 902, if either there is not reagent fixed at the sampling portion 904b (such as is the reactive agent 102, or similar, as previously discussed) or if the sampling portion 904b is capable of sitting/storing in a powder, liquid, or similar matter as the reagent material and collecting/retaining a sufficient portion of that reagent material when the sampling portion 904b is removed from the vial 902 for testing.

In use, the cap 906 is removed from the vial 902, and the sampling portion 904b with disposed reagent 908 (or, as applicable, reactive agent 102, etc.) is removed from in the vial 902. The user handles the handling portion 904a to remove the sampling portion 904b and conduct testing.

Referring to FIG. 10, another test kit 1000 includes a vial 1002 and cap 1006 to enclose a reagent 1008, for example, a liquid, powder, crystalline or other form. A test strip 1004 is external to the vial 1002 and cap 1006 enclosure, when testing is not performed. The test strip 1004 is that of the prior discussion and/or another design which allows capture of some of the reagent 1008 whenever the test strip 1004 is dipped into the reagent 1008 in the vial 1002. For example, the test strip 1004 can wick the reagent 1008 at an end or can attach or affix to reagent 1008 at an end of the strip 1004, for example, by glue, adherent, or similar means.

In use, the cap 1006 is removed from the vial 1002. The test strip 1004 is handled at an end opposite the intended testing end. The testing end of the test strip 1004 is then dipped into the open vial 1002 and into the reagent 1008. The test strip 1004 is removed from the vial 1002, with the reagent 1008 intact thereon at the testing end of the strip 1004. Testing can then proceed.

Referring to FIG. 11, another alternative test kit 1100 is an enclosed pouch 1102 with an internal space 1104 for storing a liquid, powder or similar content reagent 1108. The test kit 1100 need not, but can, include a test strip (similar to those previously described). In use, the pouch 1102 is opened, for example, by tearing or cutting, to allow the content reagent 1108 to be removed from the pouch 1102. The content reagent 1108 is then applied to a test sample. The content reagent 1108, for example, can be poured onto a surface or otherwise directly located on a sample for testing.

Alternately, the pouch 1102 can be semi-permeable to a sample for testing. In such alternative, the pouch 1102 can, itself, be placed into the sample for testing and/or the sample can be directed onto the pouch 1102. The pouch 1102 in such instance will necessarily either itself provide test readings or will allow viewing of readings shown by the contents.

Test Reagent

In accordance with the foregoing, the particular test reagent is any of a wide variety of alternatives that detect the presence of ethanol alcohol in a breast milk sample. Of course, the results of testing of the breast milk sample for alcohol must not be detrimentally affected by the composition of the breast milk sample, at least not affected to the extent required for the reagent to yield appropriate alcohol test results. At least certain particular test reagents for such breast milk testing for alcohol may be known.

A particular composition of the test reagent for the foregoing test strips and test kits is an alcohol oxidase enzyme extracted from yeast, together with a peroxidase and a hydrogen donor indicator, to provide an alcohol oxidase/peroxidase reaction. This composition reacts with ethyl alcohol if present in breast milk sample, to provide a resultant change of color in the reagent. The reaction is as follows:

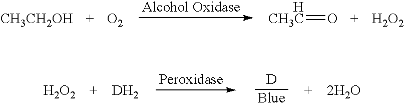

Reagent Composition:

| | | |
|---|---|---|
| Tetramethylbenzidine | 0.176 | mg |
| Alcohol Oxidase (EC 1.1.3.13) | 0.5 | IU |
| Peroxidase (EC 1.11.1.7) | 30 | IU |
| Buffer | 0.757 | mg |
| Stabilizing Proteins | 0.190 | mg |

In the test reaction, the peroxidase functions as catalyst to induce a color change in the hydrogen donor and convert the hydrogen peroxide to water.

Certain further examples of the alcohol oxidase of the reaction are described, for example, in U.S. Pat. No. 4,430,427. Certain examples of the peroxidase of the reaction, as well as other possible peroxidatively active substances, are described, for example, in U.S. Pat. No. 4,361,648. Of course, other suitable alcohol oxidase enzymes, peroxidases, and indicator agents can be employed, in keeping with the purposes herein. One manufacture of a test strip, including a reactive pad, similar to the test strip 100 of FIG. 1, is disclosed, for example, in U.S. Pat. No. 4,786,596. Of course, other compatible manufacture, in keeping with the purposes herein, can be employed.

Testing Procedure

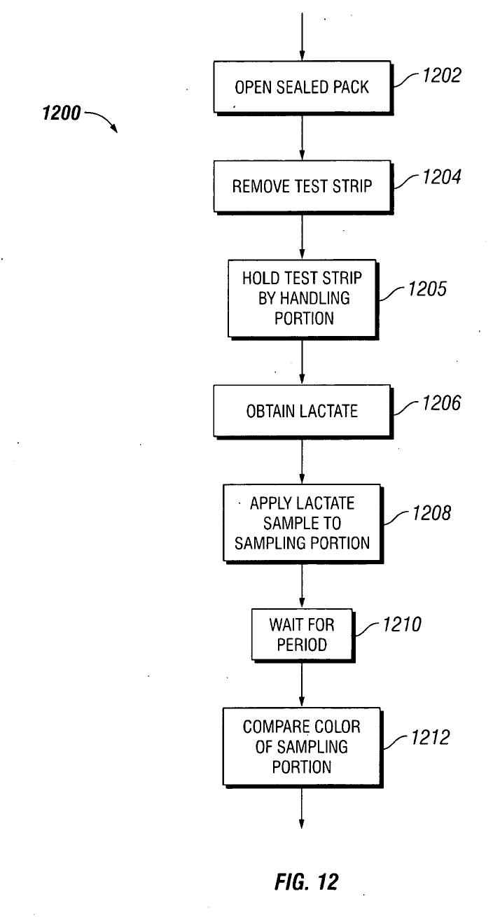

Referring to FIG. 12, a method 1200 of testing for ethanol alcohol in breast milk utilizes the foregoing test strips and test kits of the present invention. The method 1200 commences with a step 1202 of opening a pack or enclosure containing the sampling portion of the test strip. As previously mentioned, the pack or enclosure protects integrity of the test strip/reagent for testing. On opening in the step 1202, the test strip/reagent is then useable for testing.

The test strip/sampling portion/reagent is removed from the pack or enclosure in a step 1204. The testing person handles the test strip at the handling portion, to prevent affects on test integrity. In a step 1204, the user continues handling the test strip by the handling portion during the testing method 1200.

A breast milk lactate from a breast-feeding mother is obtained in a step 1206. The quantity of the breast milk lactate obtained in the step 1206 need not be great, but should be sufficient for suitably wetting the reagent of the alcohol test and for yielding a proper result of the test. The breast milk lactate is applied, in a step 1208, preferably directly (but could also be indirectly, via a container of the breast milk or other specimen thereof), onto the reagent. For example, if using a test strip/test kit as herein described, the reagent pad at the sampling portion of the strip is wetted by breast milk sample.

A waiting period in a step 1210 must then be allowed, in order for the reaction to occur or not. For example, in testing with the test reagent particularly described above, a period of at least about 2 minutes is recommended. In this wait period, the reaction of the reagent with any alcohol in the breast milk takes place, and also the indicator is triggered therein to yield a visible result.

In a step 1212, the visible result is viewed and compared to detect any change indicative of alcohol presence in the breast milk lactate. If there is not any such indicative change, then there is usually little or no alcohol (neither ethanol alcohol or certain other alcohols, for example, alcohols of less than 5 carbons using the test reagent particularly described above). On the other hand, if there is an indicative change, then usually there is some presence of alcohol (ethanol or other, for example, alcohol of less than 5 carbons using the test reagent) in the breast milk lactate.

In alternatives, quantities of alcohol in breast milk can be estimated from the viewable results. For example, a more significant or pronounced indicative change can signal greater alcohol concentration in the breast milk; whereas, a less significant or less pronounced change can signal a lesser alcohol concentration of the milk. Data (for example, obtainable through other more exact and precise laboratory testing means than those described herein, as will be known to those skilled in the art) is corresponded with varied viewable results from different alcohol-breast milk concentrations employing the testing described herein, and can be compiled as average concentration readings, to provide charts, color tabs, or other comparative devices, for estimation of concentration levels based on the viewable results of the testing described herein.

Various substances may interfere with the accuracies obtained via the testing and test strips/kits herein. Moreover, because it is expected that non-laboratory trained individuals may be performing the testing with the test strips/kits, subjective determinations by those individuals of results can be varied. In general, with the particular test reagent described herein, the following substances, for example, may affect integrity of test results: peroxidases, strong oxidizers, reducing agents (e.g., ascorbic acid, tannic acid, pyrogallol, mercaptans and tosylates), billirubin, L-dopa, L-methyldopa, metampyrone, and others. Further, other particular medications, diets, metabolism and other individualized characteristics of the breast-feeding mother can potentially affect results. Generally, however, for the purposes of most mothers, the testing, test strips, test kits and other features and aspects herein will generally provide a fair indication of alcohol in breast milk lactate, to allow those mothers to decide if there is any concern with feeding the breast milk so tested to infants.

### EXAMPLES

A test strip having a handling portion and a sampling portion, with a test reagent disposed on the sampling portion, was obtained, for example, from Chematics, Inc. P.O. Box 293, North Webster, Ind., USA 46555. A breast-feeding mother consumed a bloody mary and a glass of white wine at brunch during the period 11 am-1 pm. At about 4 pm that same day, a sample of breast milk lactate was taken from the mother's breast. The sample was disposed directly from the breast onto the reagent of the sampling portion. At least 2 minutes were waited for testing reactions to occur. The mother observed the test results, and the test results indicated no change in color of reagent. This indicated to the mother that there was no presence of ethanol alcohol (from the earlier consumption) in the breast milk at that time. The mother thereafter immediately again performed a second testing with a new, unused but identical test strip. The mother obtained a next sample of breast milk lactate from her breast. The sample was contacted with the reagent for a period of at least 2 minutes. The reagent of the second test strip did not change color, and the mother observed that the second test results also indicated lack of presence of ethanol alcohol in the breast milk at that time.

The same mother, three days thereafter, consumed a beer and two and a half glasses of wine during the period between 7 pm-10:30 pm of that day. At 12:30 am the next morning, the mother remaining awake during the period from 10:30 pm to 12:30 am, conducted a next test of her breast milk lactate. In the test, an identical, but new and unused, test strip, like those used in the prior tests, was employed. The mother contacted breast milk then obtained by her with the reagent. After a wait period of at least 2 minutes, the mother observed some darkening change in color of the reagent of the test strip. The mother observed that the test result indicated a presence of ethanol alcohol in her breast milk at that time. The mother compared the resulting color of the reagent to a comparison tab provided with the packaging of the test strip, also obtained from Chematics, Inc. (a sample of the comparison tab is included in Appendix A hereto). Based on the mother's comparative observance, the mother estimated that her breast milk did contain some alcohol, and the mother estimated that alcohol concentration of the breast milk to be about 0.3% per the comparison tab. The mother then slept until about 6 am. At 6 am, the mother next tested her lactate then obtained, using another identical, but new and unused, test strip. The mother wetted the reagent of the test strip with the breast milk. After at least 2 minutes, the mother observed the reagent of the test strip and observed no change in color, indicating to the mother the lack of presence of alcohol in her breast milk at that time.

The same mother, again, ten days thereafter, consumed alcoholic beverages. The mother did not account for the number or type of alcoholic drinks she consumed, however, the mother states she began drinking the beverages at about 7 pm that evening and continued casual drinking throughout the evening until about 1:30 am the next morning. The mother slept and woke to test at about 7 am. At 7 am, the mother obtained breast milk from her breast. The mother contacted the breast milk and the reagent of an identical, new and unused, test strip. The mother waited 2 minutes to observe the test results. The reagent color that was observed appeared dark, having changed color significantly. The mother observed that the test result indicated the presence of substantial alcohol in her breast milk at the time, on the order of about 0.3% or more. Later in the day, at about 10 am, the mother again conducted the same test in substantially the same manner. The result of the test continued to indicate presence of substantial alcohol in the mother's breast milk at that time. The mother's comparison to the comparison tab approximations indicated the breast milk contained on the order of about 0.3% alcohol. Thereafter, on the same day at about 2 pm, the mother again tested her breast milk in similar manner. The mother observed that the result of the test indicated lack of presence of alcohol in the mother's breast milk taken at that time, the reagent having not changed in color to give other/different indication.

Another breast-feeding mother also tested her breast milk lactate for alcohol presence. One evening, this breast-feeding mother consumed 4 glasses of wine at dinner during the period 7:30 pm-9:30 pm. At about 10:30 pm that same night, this mother tested her breast milk lactate. The sample was disposed directly from the breast onto the reagent of the sampling portion. At least 2 minutes were waited for testing reactions to occur. The mother observed the test results, and the test results indicated the breast milk contained on the order of about 0.3% alcohol. At 5 am the next morning, the mother again tested her breast milk in similar manner. The testing strip indicated to the mother that there was no presence of ethanol alcohol (from the earlier consumption) in the breast milk at that time.

The same mother, the next evening, consumed two and a half glasses of wine during the period between 8:30 pm-10:30 pm of that day. At 11 pm that same evening, the mother contacted breast milk then obtained by her with the reagent. The mother observed that the test resulted in a presence of ethanol alcohol in her breast milk contained on the order of about 0.3% alcohol. To show a comparison with the concentration of alcohol in her breast milk, at that same time, a saliva test was also done, indicating her blood alcohol level was 0.08%.

To compare her results in a separate sequence of testing, one evening 3 weeks later, this breast-feeding mother consumed 2 martinis and 2 beers during the period 7 pm-10 pm. At about 12:00 am that same night, this mother pumped and tested her breast milk lactate. The sample was disposed directly from the breast onto the reagent of the sampling portion. At least 2 minutes were waited for testing reactions to occur. The mother observed the test results, and the test results indicated the breast milk contained on the order of about 0.08% alcohol. The next evening, the mother again consumed alcoholic beverages in the form of 4 beers during the period 5 pm-7 pm. Two hours later, at 9 pm, she then tested her breast milk in a similar manner as before. The testing strip indicated to the mother that there was a presence of ethanol alcohol (from the earlier consumption) in the breast milk at that time, indicating a level of 0.3%.

Other alternatives are possible in keeping with the foregoing and all such alternatives are included herein. For example, different support or carriers can be employed for the test strip. Also, the particular active reagent for testing can be selected from among the various alternatives, in keeping with the purposes herein. Test strips and test kits can have a variety of forms/styles, depending on desired aspects. Comparison tabs or other color or indicator estimation measures can be derived and utilized. Additionally, the test strips, kits, and methods can be employed solely or primarily for purposes of detecting a presence of any alcohol in breast milk lactate, versus obtaining an estimated concentration. It is expected that with certain mothers, given wide variation in lifestyles, diets, metabolism, activities, and consumption, there are certain instances that may be more or less susceptible to obtaining accurate results of the breast milk-alcohol testing. Further, certain drugs, medications, and environments could impact test results, either adversely or beneficially. Variations in reagent compositions, as well as particular components of the compositions, can yield different results. Multiple testing via a varied assortment of such compositions could, for example, be conducted in each testing instance, in order to obtain more verifiable outcomes. Of course, as has been described herein with several examples and embodiments, more generalized concepts herein can be employed in testing for presence of alcohol in mothers' breast milk under differing and other conditions.

In the foregoing specification, the invention has been described with reference to specific embodiments. However, one of ordinary skill in the art appreciates that various modifications and changes can be made without departing from the scope of the present invention as set forth in the claims below. Accordingly, the specification and figures are to be regarded in an illustrative rather than a restrictive sense, and all such modifications are intended to be included within the scope of the present invention.

Benefits, other advantages, and solutions to problems have been described above with regard to specific embodiments. However, the benefits, advantages, solutions to problems and any element(s) that may cause any benefit, advantage, or solution to occur or become more pronounced are not to be construed as a critical, required, or essential feature or element of any or all the claims. As used herein, the terms “comprises”, “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

1. A test reagent for detecting alcohol, comprising:
alcohol oxidase reactive in the presence of alcohol in breast milk and oxygen, to yield acetaldehyde and peroxide;
hydrogen donor indicator having a visible color when not subjected to oxidizing agent, in sufficient amount for visible observance; and
peroxidase reactive in the presence of acetaldehyde and peroxide, to yield oxidizing agent, in sufficient amount to induce change to the visible color of the hydrogen donor indicator.

2. The test reagent of claim 1, wherein the alcohol oxidase is more selective for reactivity to alcohol, as compared to selectivity for components of breast milk not including alcohol.

3. The test reagent of claim 2, wherein the hydrogen donor indicator is tetramethylbenzidine.

4. A method of testing breast milk lactate for presence of alcohol, comprising the steps of:
providing a sealed package containing a reactive agent;
opening the sealed package;
obtaining the lactate;
contacting a reactive agent with the lactate, the reactive agent being selective to alcohol versus components for breast milk not including alcohol;
observing a visible result of the step of contacting, and
determining that (i) alcohol is present in the lactate, if the step of observing indicates change to the reactive agent as the visible result, and (ii) alcohol is not present in the lactate, if the step of observing indicates no change to the reactive agent as the visible result;
wherein the reactive agent is for one-time use in the steps;
wherein the sealed package and the reactive agent are suitable for disposal after use;
wherein an individual non-professional performs the steps of opening, obtaining, contacting, observing and determining in other than a clinical laboratory.

5. The method of claim 4, further comprising the step of waiting a period of at least 2 minutes after the step of contacting before the step of observing.

6. The method of claim 5, further comprising repeating the steps of obtaining, contacting, waiting, observing, and determining at least once immediately after the first steps.

7. A method of testing of human breast milk for presence of ethanol alcohol, comprising the steps of:
enclosing a reactive agent in a sealed hand-openable package, the reactive agent is selectively reactive with any alcohol in human breast milk lactate;
obtaining the sealed package containing the reactive agent;
opening the sealed package to access the reactive agent;
lactating by a mother to obtain a breast milk lactate sample promptly after the step of opening;
contacting the reactive agent with the sample by the mother promptly after the step of lactating; and
observing a visible result of the step of contacting by the mother, the result is changed visible color of the reactive agent if alcohol is present in the human breast milk and is unchanged visible color of the reactive agent otherwise.

8. The method of claim 7, wherein the reactive agent comprises:
alcohol oxidase reactive in the presence of alcohol in breast milk and oxygen, to yield acetaldehyde and peroxide;
hydrogen donor indicator having a visible color when not subjected to oxidizing agent, in sufficient amount for visible observance; and
peroxidase reactive in the presence of acetaldehyde and peroxide, to yield oxidizing agent, in sufficient amount to induce change to the visible color of the hydrogen donor indicator

9. A test kit for detecting presence of alcohol in human breast milk, comprising:
substrate test strip;
alcohol detector of the strip; and
visible agent for changing color in the presence of alcohol on the alcohol detector.

10. The test kit of claim 9, further comprising:
sealed package enclosing the strip, alcohol detector and visible agent when not testing.

11. The test kit of claim 10, wherein the sealed package is semi-permeable to breast milk.

12. A test kit for detecting presence of ethanol alcohol in human breast milk, comprising:
alcohol oxidase reactive in the presence of ethanol alcohol in breast milk and oxygen;
indicator having a visible color when not subjected to oxidizing agent, in sufficient amount for visible observance; and
peroxidase reactive in the presence of acetaldehyde and peroxide, to yield oxidizing agent, in sufficient amount to induce change to the visible color of the hydrogen donor indicator.

13. The test kit of claim 12, further comprising:
sealed package enclosing the alcohol oxidase, the indicator, and the peroxidase when not testing.

14. The test kit of claim 13, further comprising:
carrier strip for the alcohol oxidase, indicator, and peroxidase, in combination.

15. A method of testing human breast milk for presence of ethanol alcohol, comprising the steps of:
providing a sealed pack containing a test strip for one-time general consumer use by an individual outside of a laboratory, the test strip having a reactive component and a handling component, the handling component capable of hand manipulation by the individual without hand contact of the reactive component;
opening the sealed pack by hand by the individual outside of a laboratory;
handling the test strip via the handling component by the individual outside of a laboratory;
lactating the breast milk by the individual outside of a laboratory;
contacting the breast milk with the reactive component of the test strip by the individual outside of a laboratory;
observing outside of a laboratory a result from the step of contacting to detect a visible color change of the reactive component as indicative of ethanol alcohol presence in the breast milk;
waiting for at least about two minutes after the step of contacting before the step of observing; and
disposing of the test strip outside of a laboratory after the step of observing.

16. (canceled)

17. (canceled)

18. The product test strip in the sealed package of claim 15.

19. The product test strip containing the reactive component from the step of contacting of claim 15.
